(19) United States
(12) Patent Application Publication
(10) Pub. No.: US 2013/0061339 A1
(43) Pub. Date: Mar. 7, 2013

(54) METHOD OF DIAGNOSING TRICHOTILLOMANIA AND SIMILAR DISORDERS IN HUMANS AND RODENTS

(71) Applicants: Joseph Garner, Brisbane, CA (US); Amy C. Lossie, Lafayette, IN (US); Giovana de Lima Toccafondo Vieira, Lafayette, IN (US)

(72) Inventors: Joseph Garner, Brisbane, CA (US); Amy C. Lossie, Lafayette, IN (US); Giovana de Lima Toccafondo Vieira, Lafayette, IN (US)

(73) Assignee: Purdue Research Foundation, West Lafayette, IN (US)

(21) Appl. No.: 13/566,989

(22) Filed: Aug. 3, 2012

Related U.S. Application Data
(60) Provisional application No. 61/514,779, filed on Aug. 3, 2011.

(51) Int. Cl. G01N 33/70; A61P 25/00; A01K 67/027; A61K 31/198

(52) U.S. Cl. 800/9; 436/94; 435/4; 514/562

(57) ABSTRACT

The present disclosure provides a method of diagnosing neurological disorders including for example, impulse control disorders, such as barbering and trichotillomania using biomarkers such as reductive capacity of urine and 8-OH-dG concentration. Still other disorders that can be diagnosed based on the measurements of makers for oxidative stress include autism and Parkinson's disease.

### PRIORITY CLAIM

This application claims the benefit of U.S. provisional patent application No. 61/514,779 filed on Aug. 3, 2011, which is hereby incorporated by reference in its entirety.

### FIELD OF THE DISCLOSURE

The present disclosure relates to a method for diagnosing disorders involving the cortico-striatal circuits, and/or involving neurological pathology resulting from oxidative stress. Examples include but are not limited to: autism, impulse control disorders, and Parkinson's disease. More particularly, the present disclosure relates to the use of reactive oxygen species and DNA oxidation as an indicator for hair and feather pulling diseases in humans and other animals.

### BACKGROUND AND SUMMARY

Barbering is an abnormal repetitive behavior commonly seen in laboratory mice, where a “barber” mouse plucks hair from its cage-mates or itself, in idiosyncratic patterns, leaving the cage-mates with patches of missing fur and/or whiskers. It is not a dominance behavior. Exemplary mice displaying patches of missing fur and/or whiskers can be seen in FIGS. 1-3A. FIG. 3B shows a mouse not displaying patches of missing fur and/or whiskers.

Trichotillomania (TTM) is a human impulse control disorder characterized by compulsive hair pulling. It is one of the most common mental disorders in women, affecting 3-5% of the female population. Several lines of evidence validate barbering as a model of TTM, and barbering may also model hair and feather pulling in other species. [1].

It has been reported that N-acetylcysteine (NAC) is very effective in treating TTM. [2]. NAC is a food-additive and is a precursor to glutathione. In a randomized double-blind placebo study with 50 trichotillomania patients, NAC reduced symptoms in 56% of the patients. [2].

A further understanding of the changes in the brain that lead to TTM are desirable. Additionally, methods for detection of TTM as patients are getting ill are desirable in order to establish a screening and prevention strategy. The use of a barbering model for TTM may assist in understanding why NAC works, if it can be used to protect people before they get ill, and if those populations who NAC will help and who it will not can be predicted.

Accordingly, there exists a need for methods for predicting the onset of impulse control disorders, including barbering behavior and TTM. Some aspects of the invention disclosed herein address this need.

In some aspects of the present disclosure, biomarkers in urine are used to predict the onset of barbering. The system of the present disclosure is well suited for use in predicting the onset of barbering and how well a subject responds to NAC.

In some of these embodiments, a method for diagnosing disease has been provided, the method comprising the step of measuring the ‘reductive capacity’ in a sample of bodily fluid from a patient, wherein an elevated level of said reductive capacity indicates a need for treatment of an impulse control disorder. In some other embodiments, the method further comprises the step of treating the patient for an impulse control disorder. In some further embodiments, the method further comprises the step of administering at least one dose of a compound to the patient, and in some embodiments the compound is N-acetylcysteine.

In some of these embodiments of the method, the patient is a human, and in some further embodiments the impulse control disorder is trichotillomania (TMM). In some other of these embodiments, the patient is a mouse, and in some further embodiments the impulse control disorder is barbering behavior.

In some of these embodiments, the bodily fluid is urine. In some other of these embodiments, the bodily fluid is whole blood. In still other of these embodiments, the bodily fluid is spinal fluid. In yet still other of these embodiments, the bodily fluid is blood plasma.

In some of these embodiments, the measuring a reductive capacity step further includes measuring 8 hydroxy-2-deoxyguanoisine (8-OH-dG) levels in the bodily fluid, and/or its ratio to free antioxidant (as measured by reductive capacity), and in some further embodiments the elevated level of said reductive capacity includes an elevated level of 8-OH-dG. In other further embodiments, the elevated level of 8-OH-dG is about 8 pg 8-OH-dG/mM antioxidant or above.

In some of these embodiments, an animal model for trichotillomania is provided, the model comprising a mouse exhibiting barbering behavior and an elevated level of reductive capacity in at least one bodily fluid of the mouse.

In some of these embodiments, a method for diagnosing barbering in mice is provided, the method including collecting a urine samples from a mouse and measuring the standardized DNA oxidation of the urine, where the standardized DNA oxidation is the weight in picograms of 8-OH-dG/per millimole of antioxidant; wherein a standardized DNA oxidation of about 8 pg 8-OH-dG/mM antioxidant or greater is consistent with the exhibition of trichotillomania and/or related impulse control behaviors in the patient.

Some of these embodiments include methods for diagnosing disease behavior in humans wherein the method including collecting a urine sample from a human; and measuring the standardized DNA oxidation products in the urine, wherein a standardized DNA oxidation level, measured as ng 8-OH-dG/mg creatinine, near the upper 95% confidence interval for the general population, is consistent with the exhibition of trichotillomania and/or related impulse control behaviors, autism or Parkinson's disease in the patient.

In some of these embodiments, a method screening for a compound to treat impulse control disorder is provided, the method screening comprising the steps of administering at least one dose of a compound to a mammal, and measuring a change in the reductive potential of at least one bodily fluid from the animal, wherein said animal is susceptible to an impulse control disorder and wherein said compound reduces the reductive potential measured in the bodily fluid of the mammal. In some further embodiments, the mammal is a mouse, wherein the mouse is susceptible to developing the impulse control disorder barbering behavior. In other further embodiments, the mammal is a human being, wherein said human being is susceptible to developing the impulse control disorder trichotillomania.

### BRIEF DESCRIPTION OF THE DRAWINGS

The above-mentioned and other features of the present disclosure will become more apparent and will be better understood by reference to the following description of embodiments of the present disclosure taken in conjunction with the accompanying drawings, wherein:

FIG. 1. Photographs of mice from which hair and/or whiskers have been plucked.

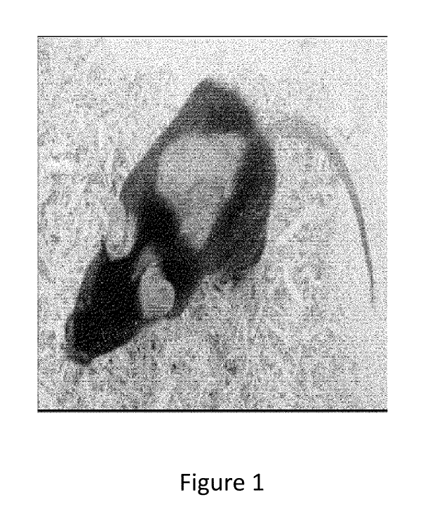

FIG. 2A Photographs of a mouse (top of the picture) from which hair and/or whiskers have not been plucked.

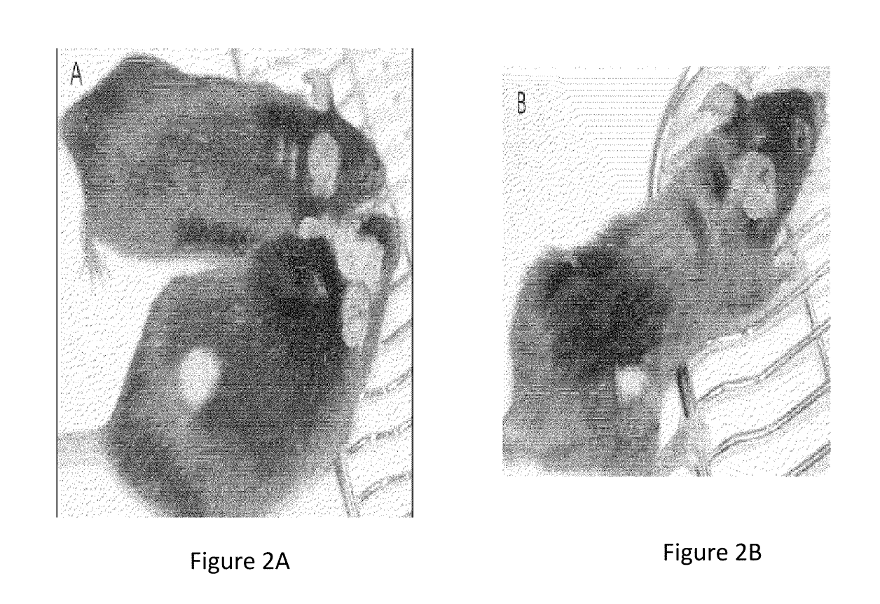

FIG. 3. Photographs of mice from which hair and/or whiskers have been plucked.

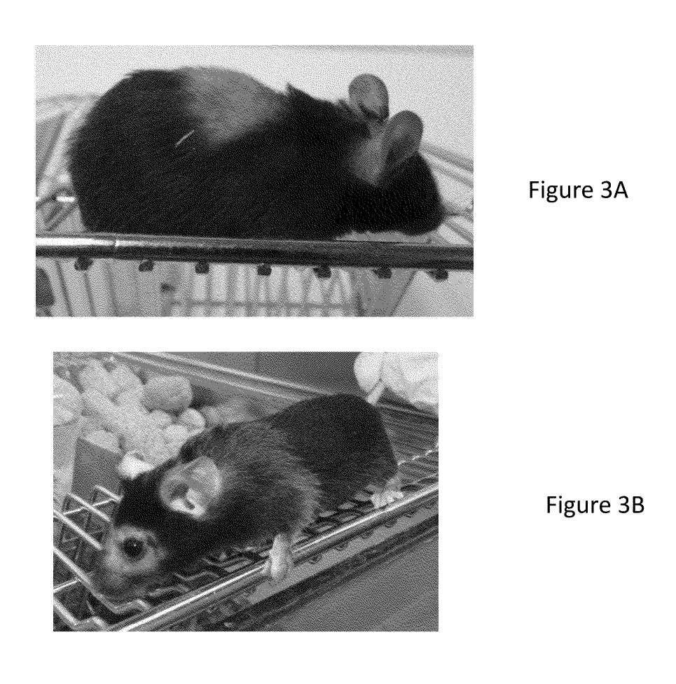

FIG. 4. A schematic view of a causal pathway for barbering.

FIG. 5. A schematic view of the production of glutathione from N-acetylcysteine.

FIG. 6. A schematic view of pathways from diet, stress, and NAC to TTM and barbering.

FIG. 6A A schematic views of pathways from diet, stress, and NAC to TTM and barbering.

FIG. 7. Illustrates the relative reductive capacity of urine from barbers and non-barbers.

FIG. 8. Illustrates the urinary total antioxidant capacity of soccer players at different points in the season including (1) pre-season, (2) early in-season, and (3) start of end-season.

FIG. 9. Illustrates s the urinary total antioxidant capacity of different groups of soccer players including (1) professionals, (2) amateurs, and (3) recreational players.

FIG. 10. Illustrates the likelihood of a mouse becoming a barber as a function of the standardized reductive capacity of the mouse's urine before treatment.

FIG. 11. Illustrates the proportion of mice barbering after 24 weeks of treatment for mice that both were and were not barbers at the start of treatment.

FIG. 12. Illustrates the standardized reductive capacity after treatment for mice that both were and were not barbers at the start.

FIG. 13. Illustrates the likelihood of a mouse becoming a barber as a function of the standardized reductive capacity of urine before treatment.

FIG. 14. Illustrates the likelihood of a mouse becoming a barber as a function of the standardized DNA oxidation.

Corresponding reference characters indicate corresponding parts throughout the several views. Although the drawings represent embodiments of the present disclosure, the drawings are not necessarily to scale and certain features may be exaggerated in order to better illustrate and explain the present disclosure.

### DETAILED DESCRIPTION OF THE DISCLOSURE

The embodiments disclosed herein are not intended to be exhaustive or limit the disclosure to the precise forms disclosed in the following detailed description. Rather, the embodiments are chosen and described so that others skilled in the art may utilize their teachings.

As used herein, unless explicitly stated otherwise or clearly implied otherwise the term ‘about’ refers to a range of values plus or minus 10 percent, e.g. about 1.0 encompasses values from 0.9 to 1.1.

As used herein, unless explicitly stated otherwise or clearly implied otherwise the terms ‘therapeutically effective dose,’ ‘therapeutically effective amounts,’ and the like, refers to a portion of a compound that has a net positive effect on the health and well-being of a human or other animal. Therapeutic effects may include an improvement in longevity, quality of life and the like these effects also may also include a reduced susceptibility to developing disease or deteriorating health or well-being. The effects may be immediate realized after a single dose and/or treatment or they may be cumulative, and realized after a series of doses and/or treatments.

Reactive Oxygen Species (ROS), also known as free radicals, are a normal but deadly byproduct of glucose metabolism in every cell. ROS are produced as a consequence of normal aerobic metabolism, which in turn is regulated by the Hypothalamic-Pituitary-Adrenal (HPA) and Sympathetic-Adrenal-Medullary (SAM) axes. Thus a variety of factors, including diet and chronic stress, elevate ROS in the body. The cells of the human brain consume about 20% of the oxygen utilized by the body but constitute only 2% of the body weight. Consequently, reactive oxygen species which are continuously generated during oxidative metabolism will be generated in high rates within the brain.

While ROS are produced as a product of normal cellular functioning, excessive amounts can cause harmful effects. Unstable free radicals species attack cellular components causing damage to lipids, proteins and DNA, which can initiate a chain of events resulting in the onset of a variety of diseases. Nerve cells are particularly vulnerable to oxidative damage from ROS. Oxidative damage has been implicated in the pathogenesis of several psychiatric disorders such as Parkinson's Disease and Alzheimer's Disease.

The production of free radicals can be increased by diet, psychological and physiological stress, and hormonal changes. If a cell is making more free radicals than it has antioxidants to neutralize them, then the cell suffers oxidative stress.

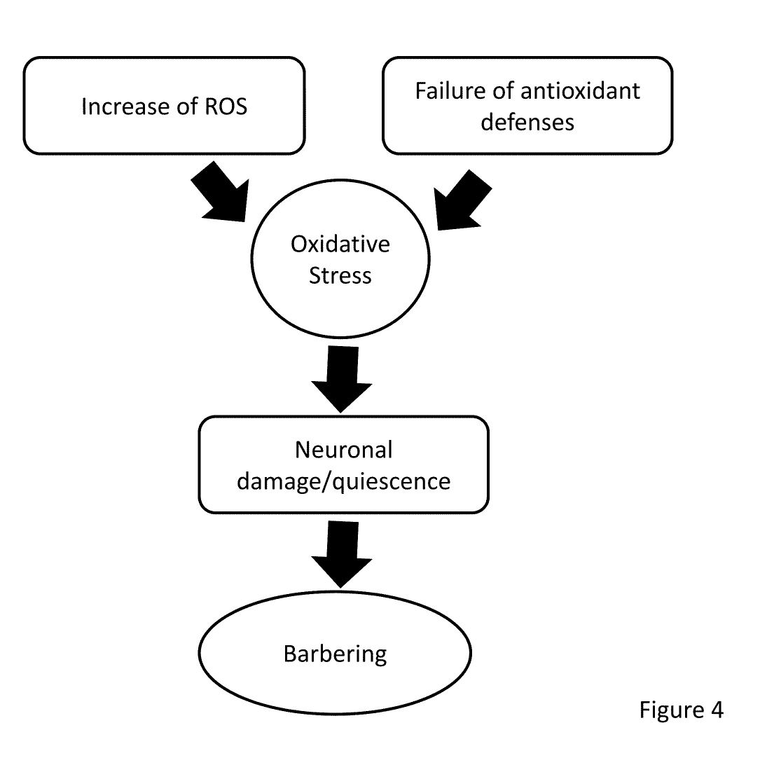

FIG. 4 illustrates a model in which barbering is caused by neuronal damage or quiescence as a result of oxidative stress caused by multifactorial sources for elevated ROS and/or a failure to activate antioxidant defenses. Nerve cells are particularly vulnerable to oxidative stress because of the amount of glucose they use. If the cells cannot cope with the oxidative stress, the cells can either die (apoptosis) or go into a sleep mode and stop firing (quiescence). Coincidentally the brain areas involved in TTM (the cortico-striatal circuits) are most sensitive to oxidative stress right around the peak age of onset in puberty.

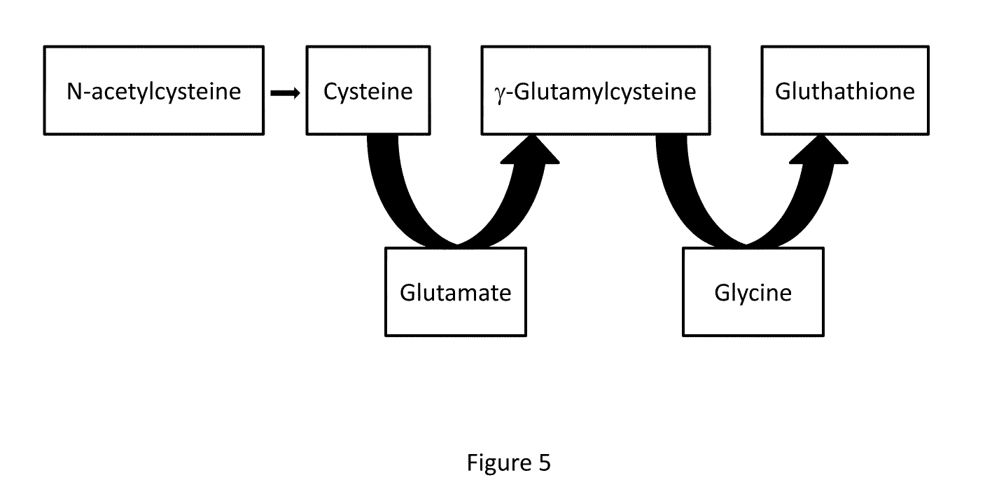

Cells respond to oxidative stress by activating defenses which produce special antioxidants. Glutathione is the main antioxidant produced to defend the brain. N-Acetylcysteine (NAC) is a food-additive and is a precursor to glutathione. FIG. 5 illustrates the production of glutathione from NAC. NAC, which is very effective in treating TTM [2] is the most important ingredient in this defense. In a randomized double-blind placebo study with 50 trichotillomania patients, NAC reduced symptoms in 56% of the patients.

In another study, Dufour et al. fed mice with a diet that elevated blood glucose and induced insulin release. [3]. Dufour et al. reported an increase in barbering severity on those animals. This study inherently increased ROS production in the mice by elevating blood glucose, and the increase in barbering severity observed is consistent with barbering being caused by neuronal damage or quiescence as a result of oxidative stress caused by elevated ROS and/or a failure to activate antioxidant defenses.

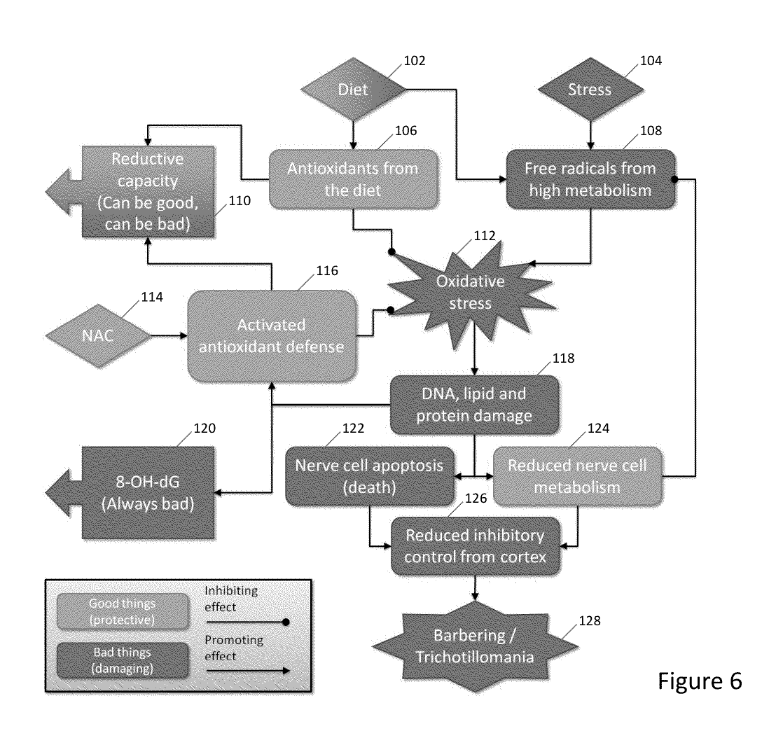

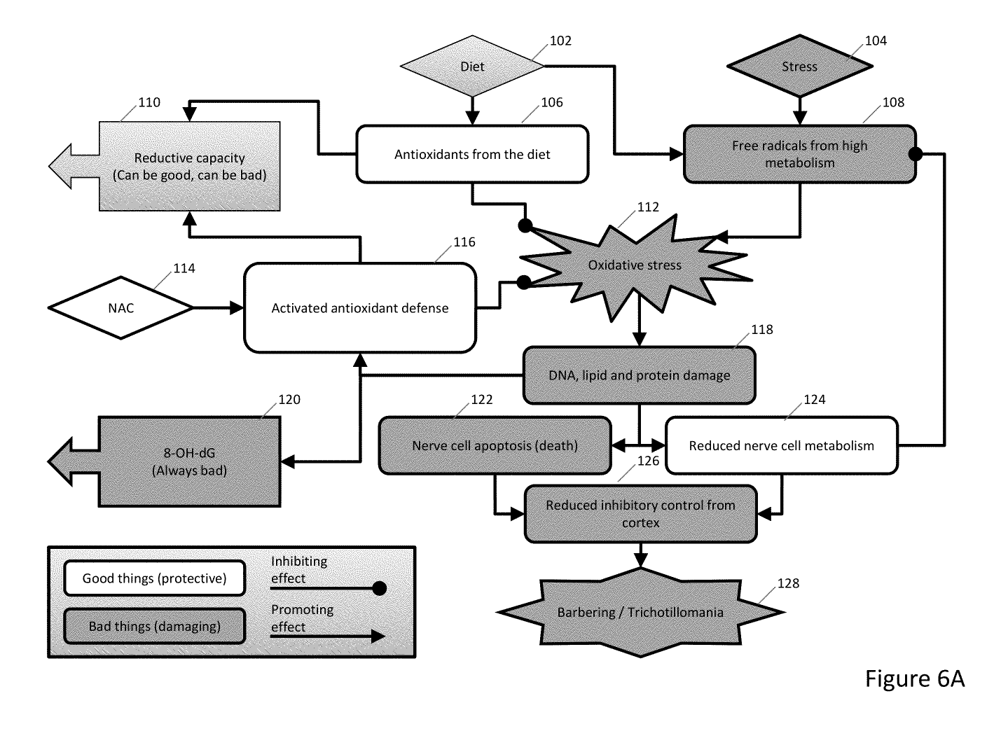

TTM and barbering is thought to be caused when cells in the brain areas implicated in TTM and barbering experience oxidative stress, in some patient the cells cannot defend themselves and enter apoptosis or quiescence. FIGS. 6 and 6A illustrate the relationships between diet, stress, NAC, and barbering/TTM as predicted by this model, including various inhibiting and promoting effects.

Ongoing oxidative stress promotes damage to DNA. ROS damage to DNA produces, among other things, 8 hydroxy-2-deoxyguanoisine (8-OH-dG). The level of 8-OH-dG can be measured in urine as a biomarker for the level of DNA damage done by ROS due to oxidative stress.

Referring again to FIGS. 6 and 6A, inhibiting effects are indicated by lines terminating in circles, while promoting effects are indicated by lines terminating in arrows. Diet 102 promotes both antioxidants from the diet 106 and free radicals from high metabolism 108. Stress 104 promotes free radicals from high metabolism 108. Antioxidants from the diet 106 inhibit oxidative stress 112, while free radicals from high metabolism promote oxidative stress 112. The addition of NAC 114 promotes the activated antioxidant defense 116, which inhibits oxidative stress 112. Both antioxidants from the diet 106 and activated antioxidant defense 116 have a promoting effect on reductive capacity 110. Oxidative stress 112 has a promoting effect on DNA lipid and protein damage 118. Elevated 8-OH-dG 120 is a highly specific biomarker of DNA damage 118. DNA, lipid and protein damage 118 has a promoting effect on nerve cell apoptosis 122 and reduced nerve cell metabolism 124. Reduced nerve cell metabolism has an inhibiting effect on free radicals from high metabolism 108. Nerve cell apoptosis 122 and reduced nerve cell metabolism both have a promoting effect on reduced inhibitory control from cortex 126, which has a promoting effect on barbering or TTM 128. Protective (good things) blocks in FIGS. 6 and 6A include blocks 106, 114, 116, and 124. Damaging (bad things) blocks in FIGS. 6 and 6A include blocks 104, 108, 112, 118, 120, 122, 126, and 128. Blocks 102 and 110 have both protective and damaging qualities.

As a result, barbers should show higher oxidative stress than non-barbers. NAC should prevent as well as cure barbering. Oxidative stress should predict how well mice respond to NAC. The onset of barbering should be associated with signs of oxidative damage to cells. Biomarkers of oxidative stress in the urine can be measured to test these predictions.

### EXAMPLES

### Example 1

### Test to Determine if Total Reductive Capacity (TAC) is a Predictive Biomarker for Barbering

The Total Antioxidant Capacity (TAC) of urine was evaluated as a predictive biomarker. TAC measures the cumulative action of all the antioxidants present in urine.

Twenty-six female C57BL/6J mice aged between 2 and 8 months were selected from our colony. The animals were housed with siblings. Each mouse was categorized as a barber or non-barber. A minimum of 0.5 ml of urine was collected by manual compression of the bladder from each mouse and the samples were then frozen at −80° C. for later analysis. The urine was analyzed for TAC and creatinine to control for urine concentration.

For statistical analysis, logistic regression was used to test whether TAC:creatinine ratio was a predictive biomarker for barbering. The analyses were blocked by cage and weight.

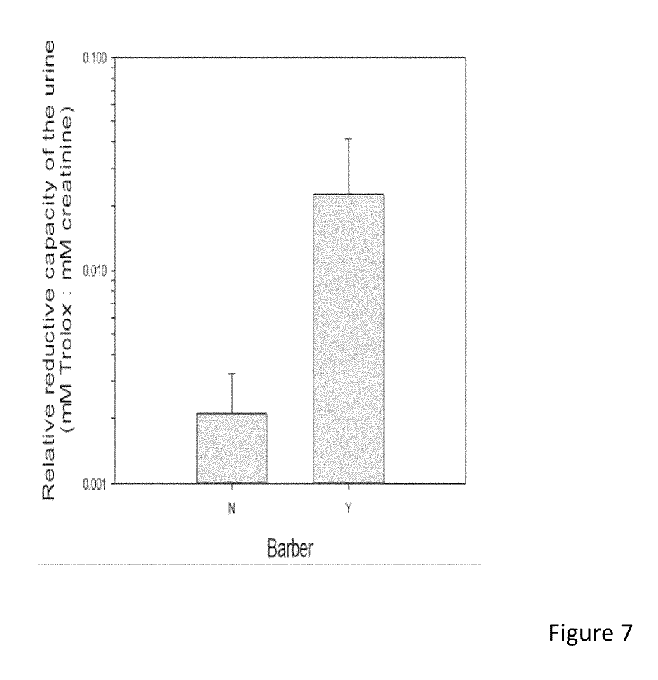

Results of the analysis can be found in FIG. 7, which shows the relative reductive capacity of urine from barbers and non-barbers. FIG. 7 illustrates the reductive capacity measured in mM of the antioxidant Trolox:mM creatinine Barbers had higher total antioxidant capacity of urine than non-barbers (LR Chi Sq=215.5; p<0.001).

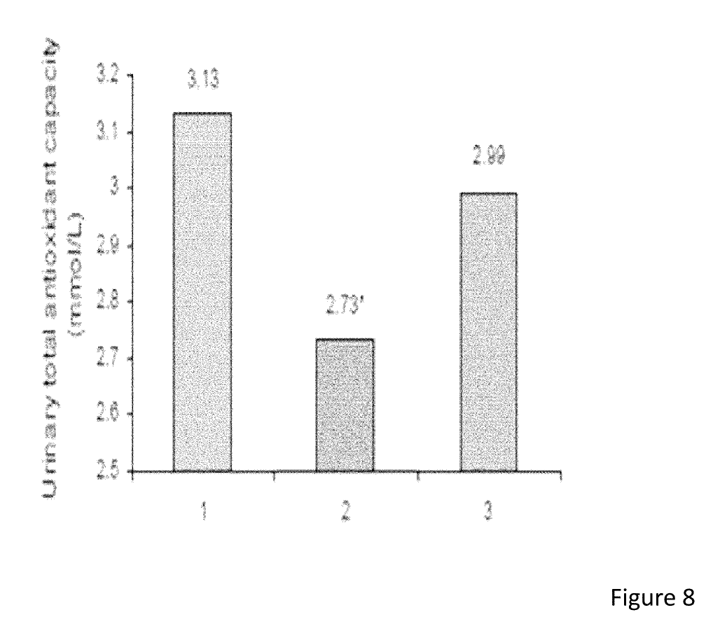

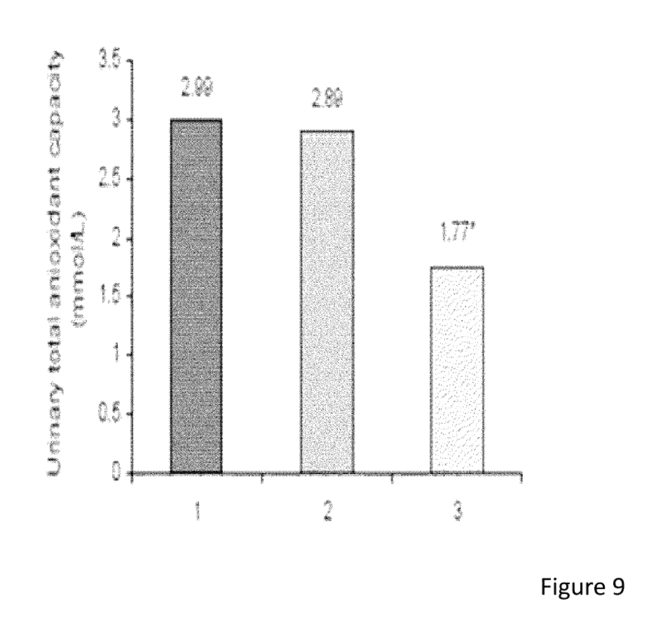

In a study by Mukherjee and Chia [4], the antioxidant capacity of urine of soccer players in different stages of playing season was followed. The results of this study can be seen in FIGS. 8 and 9. FIG. 8 shows the urinary total antioxidant capacity of soccer players at different points in the season including (1) pre-season, (2) early in-season, and (3) start of end-season. FIG. 9 shows the urinary total antioxidant capacity of different groups of soccer players including (1) professionals, (2) amateurs, and (3) recreational players.

It is conceivable that as the competition season commences, there is an increase in the volume as well as in the intensity of exercise and consequently an increase in high intensity aerobic exercise, and is possible that the recovery during this period would be insufficient in the players. Such an increase in exercise load would cause a significant increase in the oxidative stress due to increased generation of free radicals due to a higher aerobic load. In theory, this should have led to an increased antioxidant response.

As shown in FIG. 8, on the contrary, there was a significant decrease in the antioxidant capacity in the professional soccer players from the pre-season phase (1) to the early in-season phase (2) of the soccer season. Start of end-season is shown as (3). This finding can be explained based on the understanding that an excessive production of free radicals severely hampers the antioxidant defenses and causes changes in the cellular homeostasis mechanisms.

As shown in FIG. 9, the same study showed that the players at the professional (1) and amateur (2) levels had higher antioxidant capacity than the recreational (3) players, confirming that the antioxidant response parallels the increase in oxidative stress.

One possible explanation for the higher total antioxidant capacity of urine of barbers compared to non-barbers, is that barbers, like soccer the players, overproduced antioxidants as a compensatory response from the body to the overwhelming period of oxidative stress and high levels of ROS in the brain.

Conclusion: a relationship between oxidative stress and barbering behavior is confirmed, and provides a potential physiological biomarker for the disease mechanism.

### Example 2

### Tests Measuring Biomarkers of Oxidative Stress in Urine

Thirty-two female adult C57BL/6J mice (14 barbers and 18 non-barbers) were separated into cages with no barbers, and cages with at least one barber. Cages with no barbers were fed a diet to induce barbering, as per Dufour, et al. [3]. Cages with barbers were fed a regular mouse diet. Half the cages in each group had their feed supplemented with NAC, at a dose of 1 g/kg/day per mouse. Urine was collected at baseline and 24 weeks, to measure: reductive capacity, which reflects the sum of unused antioxidants from the diet, and antioxidants produced to defend the body. The level of 8 hydroxy-2-deoxyguanosine (8-OH-dG) which measures DNA damage from ongoing oxidative stress was also measured at 24 weeks only. Urine concentration was controlled by measuring creatinine.

Six animals provided urine too dilute for analysis. Barbering and patterns of hair loss were recorded every 2 weeks.

Data were analyzed using generalized linear model (GLM) and logistic regression using JMP statistical software, available from SAS, Cary N.C.

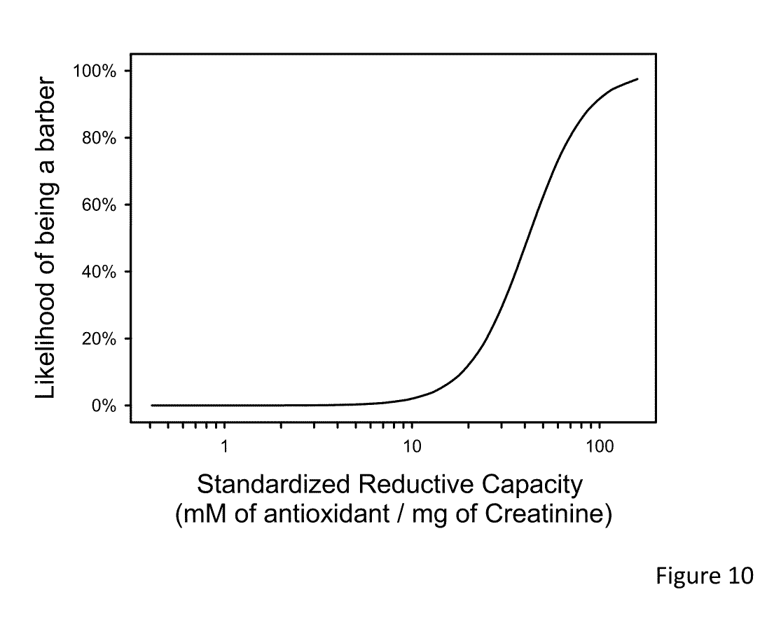

FIG. 10 shows the likelihood of a mouse becoming a barber as a function of the standardized reductive capacity of the mouse's urine before treatment. FIG. 10 illustrates that the high reductive capacity of the urine identifies barbers before treatment (P=0.0117). Mice having a standardized reductive capacity below about 10 mM of antioxidant/mg of creatinine were unlikely to become a barber. Mice having an elevated standardized reductive capacity were more likely to become barbers.

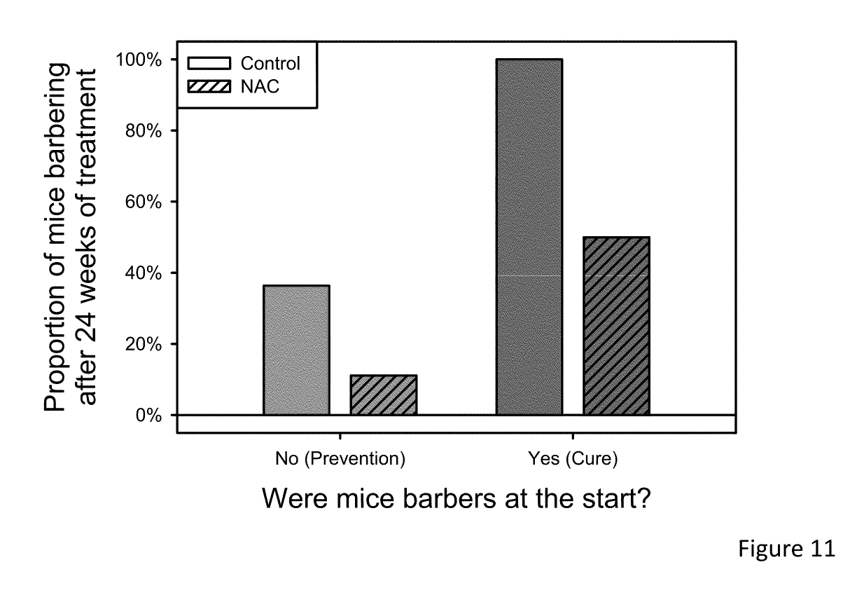

FIG. 11 shows the proportion of mice barbering after 24 weeks of treatment for mice that both were and were not barbers at the start. FIG. 11 illustrates that NAC both protects mice from become barbers and cures mice starting as barbers (P=0.0152), and does not differ in efficacy for both (P=0.3106). For mice that were not barbers at the start, fewer mice treated with NAC were barbering after 24 weeks of treatment than mice not treated with NAC, indicating a preventative effect. For mice that were barbers at the start, fewer mice treated with NAC were barbering after 24 weeks of treatment than mice not treated with NAC, indicating a curative effect.

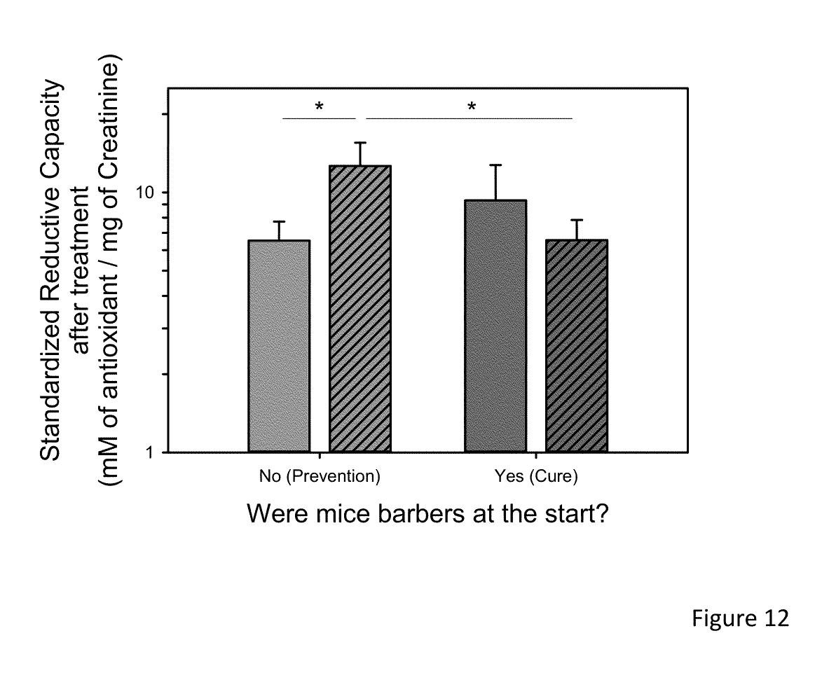

FIG. 12 shows the standardized reductive capacity after treatment for mice that both were and were not barbers at the start. FIG. 12 illustrates that NAC only increases reductive capacity in healthy mice, and does more so than in barbers (P=0.0244).

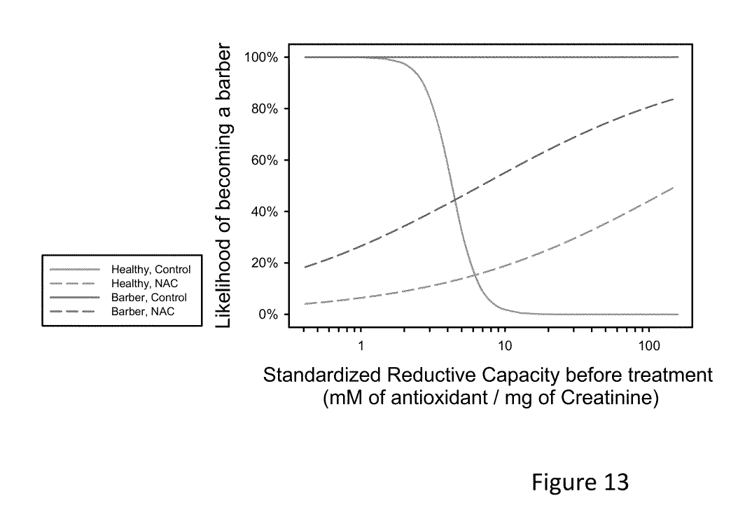

FIG. 13 shows the likelihood of a mouse becoming a barber as a function of the standardized reductive capacity of urine before treatment. FIG. 13 illustrates that low reductive capacity predicts the healthy mice which become ill, but not which mice on NAC will respond (P=0.0238).

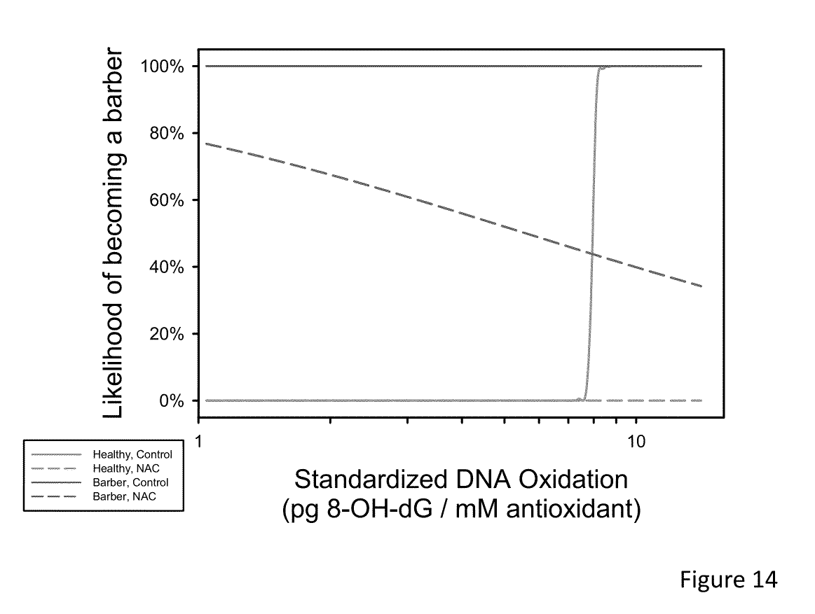

FIG. 14 shows the likelihood of a mouse becoming a barber as a function of the standardized DNA oxidation, measured as pg 8-OH-dG per mM antioxidant. FIG. 14 illustrates that elevated levels of 8-OH-dG predicts which mice became barbers during the experiment (P=0.0035). At low levels of standardized DNA oxidation, no healthy control mice became barbers. At elevated levels of standardized DNA oxidation, all healthy control mice became barbers. As illustrated in FIG. 14, an elevated level is about 8 pg 8-OH-dG per mM antioxidant or above in mice.

### Example 3

### Comparing Mouse Model to Humans

As reported by Wu, et al. [5], the normal level of 8-OH-dG in the urine of healthy humans, (given in units of ng of 8-OH-dG per mg creatinine) is 43.9+/−42.1 in females and about 29.6+/−24.5 in males. The DNA damage of a population of mice was measured in unites of ng 8-OH-dG/mg of creatinine, controlled for baseline reductive capacity. At the mean baseline reductive capacity, the mice switched from essentially 100% safe (i.e. non-barber) to essentially 100% at-risk (i.e. barber) between 344 and 380 ng 8-OH-dG/mg creatinine The level of ng 8-OH-dG/mg creatinine in this mouse population ranged from 154 to 744, with a mean of 335. Thus the mice switched to essentially 100% at-risk (i.e. barber) close to the upper 95% confidence interval of the population mean (390 ng 8-OH-dG/mg creatinine) One can therefore approximate the danger point as being the upper 95% confidence interval for a given population. Extrapolating this model to humans, in which females exhibit a mean ng 8-OH-dG/mg creatinine value of 43.9 and males exhibit a mean ng 8-OH-dG/mg creatinine value of 29.6, the danger points are approximately 138 ng 8-OH-dG/mg creatinine for human females and about 78 ng 8-OH-dG/mg creatinine for human males.

### Results

As demonstrated by the examples presented herein, barbering is a disease of oxidative stress. Reductive capacity is a biomarker for ill mice. DNA oxidation is a biomarker for onset of barbering. Reductive capacity is also a biomarker for which healthy mice will get ill in the future. Reductive capacity suggests that NAC works by reducing oxidative stress, but does not predict if NAC will work.

Barbering has been validated as a model for TTM in humans and may also model hair and feather pulling in other species. [1]. NAC has been reported as very effective in treating TTM. Based on this model, reductive capacity can be used as a biomarker for TTM and the prediction of TTM. Additionally, DNA oxidation can be used as a biomarker for the onset of TTM based upon this model.

### REFERENCES

The following listed references are expressly incorporated by reference herein. Throughout the specification, these references are referred to by citing to the numbers in the brackets [#].
[1] Garner, J. P., Weisker, S. M., Dufour, B., Mench, J. A., 2004. “Barbering (fur and whisker trimming) by laboratory mice as a model of human trichotillomania and obsessive-compulsive spectrum disorders,” Comp. Med. 54(2), 216-224.
[2] Grant, J. E., Odlaug, B. L., Kim, S. W., 2009. “N-acetylcysteine, a glutamate modulator, in the treatment of trichotilomania: a double-blind, placebo-controlled study,” Arch. Gen. Psych. 66(7), 756-763.
[3] Dufour, B. D., Adeola, O., Cheng, H. W., Donkin, S. S., Klein, J. D., Pajor, E. A., Garner, J. P., 2010. “Nutritional upregulation of serotonin paradoxically induces compulsive behavior,” Nutr. Neurosci. 13(6), 256-264.
[4] Mukherjee, S. and Chia, M., 2009. “Urinary total antioxidant capacity in soccer players,” Acta Kinesiologica 3(1), 26-33.
[5] Wu, L. L., Chiou, C.-C., Chang, P.-Y., Wu, J. T., 2004. “Urinary 8-OHdG: a marker of oxidative stress to DNA and a risk factor for cancer, atherosclerosis and diabetics” Clin. Chim Acta, 339 (1-2), 1-9.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

What is claimed is:

1. A method for diagnosing disease, the method comprising the step of:
measuring a reductive capacity in a sample of bodily fluid from a patient, wherein an elevated level of said reductive capacity indicates a need for treatment of disorders involving the cortico-striatal circuits, and/or involving neurorological pathology resulting from oxidative stress.

2. The method according to claim 1, wherein the disorder is selected from the group consisting of the impulse control disorders, autism, and Parkinson's disease.

3. The method of claim 1, further comprising the step of treating the patient for a neurological disorder.

4. The method of claim 1, further comprising the step of administering at least one therapeutically effective dose of a compound to the patient.

5. The method of claim 4, wherein the compound is N-acetyl cysteine.

6. The method of claim 1, wherein the patient is a human.

7. The method of claim 2, wherein the impulse control disorder is trichotillomania.

8. The method of claim 1, wherein the patient is a mouse.

9. The method of claim 2, wherein the impulse control disorder is barbering behavior.

10. The method of claim 1, wherein the bodily fluid is urine.

11. The method of claim 1, wherein the measuring a reductive capacity step includes measuring the standardized concentration of an oxidant per creatinine in the urine.

12. The method of claim 1, wherein the bodily fluid is selected from the group consisting of whole blood, blood plasma, and spinal fluid.

13. The method of claim 1, wherein the measuring the reductive capacity of a sample includes measuring 8-OH-dG levels in the bodily fluid and wherein an elevated level of said reductive capacity is characterized by an elevated level of 8-OH-dG in the sample.

14. The method of claim 13, wherein the elevated level of 8-OH-dG in a sample of urine from a mouse is in the range about 280-390 ng 8-OH-dG per mg of creatinine.

15. The method of claim 13, wherein the elevated level of 8-OH-dG in a sample of urine form a human female is about 138 ng 8-OH-dG per mg of creatinine and in human males is about 78 ng 8-OH-dG per mg of creatinine.

16. An animal model for trichotillomania, comprising:
a mouse exhibiting barbering behavior; and
an elevated level of reductive capacity in at least one bodily fluid of the mouse.

17. A method for diagnosing trichotillomania in a human patient comprising:
measuring the level of 8-OH-dG normalized to the amount of creatinine in the sample wherein values near the upper 95% confidence interval are predictive of trichotillomania.

18. A method screening for compounds to treat disorders of cortico-striatal circuits, and/or involving neurorological pathology resulting from oxidative stress, comprising the steps of:
administering at least one dose of a compound to a mammal;
measuring a change in the reductive potential of at least one bodily fluid from the animal, wherein said animal is susceptible to an impulse control disorder and wherein said compound reduces the reductive potential measured in the bodily fluid of the mammal.

19. The method according to claim 18, wherein the mammal is a mouse, wherein the mouse is susceptible to developing the impulse control disorder barbering behavior.

20. The method according to claim 18, wherein mammal is a human being, wherein said human being is susceptible to developing the impulse control disorder trichotillomania.
